(19) United States
(12) Patent Application Publication
(10) Pub. No.: US 2006/0246161 A1
(43) Pub. Date: Nov. 2, 2006

(54) METHOD OF MAKING MEDICAMENT FOR TREATING ANEMIA

(76) Inventors: Hongtao Xing, Hong Kong (CN); Chi Man Lee, Hong Kong (CN); Shiu Fun Pang, Hong Kong (CN)

Correspondence Address: FISH & RICHARDSON PC, P.O. BOX 1022, MINNEAPOLIS, MN, 55440-1022, US

(21) Appl. No.: 11/420,896

(22) Filed: May 30, 2006

Related U.S. Application Data
(63) Continuation-in-part of application No. 10/783,619, filed on Feb. 20, 2004.
(60) Provisional application No. 60/448,935, filed on Feb. 21, 2003.

(51) Int. Cl. A61K 36/185; A61K 31/704; A61K 31/56

(52) U.S. Cl. 424/725; 514/26; 514/169

(57) ABSTRACT

Method and pharmaceutical composition for treating anemia related symptoms. The method and formulation involve the use of one or more cucurbitacin analogs as active ingredients, for example, cucurbitacin D, which are capable of increasing hemoglobin expression, reactivating fetal or adult hemoglobin and inducing γ-globin.

### CROSS REFERENCE TO RELATED APPLICATION

This application is a continuation-in-part of U.S. application Ser. No. 10/783,619, filed Feb. 20, 2004, which claims priority to U.S. Provisional Application Ser. No. 60/448,935, filed Feb. 21, 2003, the contents of which are hereby incorporated by reference.

### FIELD OF THE INVENTION

This invention relates to a method of disease treatment and to a pharmaceutical composition. Particularly, it relates to making a pharmaceutical composition or medicament for treating anemia conditions in mammalian subjects using an extract of herbal medicine Trichosanthes or a compound of the cucurbitanin analog.

### BACKGROUND OF THE INVENTION

Cucurbitacins

Cucurbitacins were initially known as the ingredients that give the Cucurbitacae its bitter taste, and were later found to be present in plants in a number of families, such as Brassicaceae, Scrophulariaceae, Begoniaceae, Elaeocarpaceae, Datiscaceae, Desfontainiaceae, Polemoniaceae, Primulaceae, Rubiaceae, Sterculiaceae, Rosaceae, and Thymelaeaceae, where they may be either non-glycosylated or glycosylated. More recently, cucurbitacins have also been isolated from several genera of mushroom, including Russula and Hebeloma, and even from shell-less marine mollusks (dorid nudibranchs) (Chen et al., 2005). A typical purification process involves extraction of the cucurbitacins in plants or plant extracts by non-polar solvents such as hexane, petroleum ether and ethanol followed by separation of cucurbitacins by column chromatography or high-performance liquid chromatography using silica gel columns (U.S. Pat. No. 5,925,356). Traditionally, the cucurbitacins are arbitrarily divided into twelve categories. The natural cucurbitacins is a group of triterpenoid substances which are well-known for their bitterness and toxicity. Structually, they are characterized by the tetracyclic cucurbitane nucleus skeleton, namely, the 19-(10→9β)-abeo-10 alanost-5-ene (also known as 9β-methyl-19-nor lanosta-5-ene), with a number of oxygenation functionalities at different positions. They are present in many plants as β-glucosides and function as an allomone to protect the plants from herbivores (Setzer et al., 2003). Recently, cucurbitacins are also known to possess a number of potent pharmacological effects, deriving largely from their cytotoxic, anti-cancer and anti-inflammatory properties.

For example, a new cucurbitacin D analogue, 2-deoxycucurbitacin D, cucurbitacin D and 25-acetylcucurbitacin F isolated from Sloanea zuliaensis have shown cytotoxic activity against breast, lung and central nervous system human cancer cell lines (Rodriguez et al., 2003). Cucurbitacin D, E and I found in Gonystylus Keithii have been shown to be cytotoxic toward renal tumor, brain tumor and melanoma cell lines (Fuller et al., 1994). Cucurbitacin B from Picrorhiza scrophulariaeflora is known to inhibit mitogen-induced T cell proliferation (Smit et al., 2000). Cucurbitacin B and isocucurbitacin B from Helicteres Isora, Ipomopsis Aggregata and Casearia arborea have been found to have cytotoxic activity against Eagle's carcinoma of the nasopharynx in cell culture (Bean et al., 1985), human nasopharyngeal carcinoma (Arisawa et al., 1984) and National Cancer Institute (NCI) 60-cell lines of human tumor screening (Beutler et al., 2000), respectively. Cucurbitacin B, and two new cucurbitane triterpenoids, leucopaxillones A and B, isolated from Leucopaxillus gentianeus have been found to inhibit the proliferation of human lung carcinoma, epatoblastoma, breast adenocarcinoma, and kidney carcinoma cell lines (Clericuzio et al., 2004). Cucurbitacin E purified from Conobea Scoparioided has been demonstrated to have inhibitory effect towards leukocyte intergrin-mediated cell adhesion (Musza et. al., 1994). Elaeocarpus dolichostylus has been found to have cytotoxic activity towards nasopharynx carcinoma cell lines, and the active ingredient appears to be cucurbitacin F because it was present in the bio-active fraction (Fang et al., 1984). Lastly, cucurbitane triterpenoids, cayaponosides B, B3, D, D3b and C2 isolated from Cayaponia tayuya have exhibited inhibitory effect on Epstein-Barr virus activation (Konoshima et al., 1995).

Cucurbitacin D, which lacks the acetyl group at the 25-OH, is the most ubiquitous cucurbitacin known. It has also been found to antagonize the action of insect steroid hormones, and interfere with the growth of symbiotic bacteria of entomopathogenic nematodes in vitro (Barbercheck et al., 1996). Cucurbitacin D has showed significant cytotoxicity against a variety of human cancer cell lines from many independent studies, including lung cancer, human colon cancer, human oral epidermoid carcinoma, hormone-dependent human prostate cancer, human telomerase reverse transcriptase-retinal pigment epithelial cells, and human umbilical vein endothelial cells, breast (MCF-7), as well as, central nervous system (SF-268) cancer cell lines (Chen et al., 2005). It has also been found that cucurbitacin D was able to induce morphological changes of Ehrlich ascites tumor cells at low concentrations and to affect respiration, permeability, and viability of these cells at higher concentration (Duncan et al., 1996). Furthermore, cucurbitacin D has been shown to enhance capillary permeability, which is then associated with a persistent fall in blood pressure and the accumulation of fluid in thoracic and abdominal cavities in mice (Edery et al., 1961).

However, to the applicant's knowledge, at the time of the present invention it is unknown that cucurbitanins have any anti-anemia effects.

Anemia

Anemia, one of the most common blood disorders, occurs when there is a deficiency of red blood cells (RBCs) and/or hemoglobin in the body. It leads to health problems as red blood cells contain hemoglobin, which carries oxygen to the body's tissues. Anemia can cause a variety of complications, including fatigue and stress on bodily organs. Anemia is due to either excessive destruction of RBCs or blood loss, or inadequate production of RBCs. Among many other causes, anemia can result from inherited disorders, nutritional problems (such as an iron or vitamin deficiency), infections, some kinds of cancer, or exposure to a drug or toxin.

The hereditary hemoglobinopathies such as sickle cell anemia and thalassemias are among the most prevalent serious genetic disorders affecting human populations and represent a major health burden worldwide. Even nowadays, blood transfusions remain the major means to ameliorate the clinical anemia syndromes although they are only of temporary benefit. Hypertransfusion induced iron overload requires effective iron chelating therapy to improve growth and prolong survival in patients. Bone marrow transplantation can cure the disease but has not been widely performed because of risk, expense, the need for an HLA compatible donor and poor acceptance by families and patients.

Recently, the search for treatment aimed at reduction of globin chain imbalance in patients with thalassemia has focused on the pharmacologic manipulation of fetal hemoglobin (α₂γ₂; HbF). The switch from fetal hemoglobin to adult hemoglobin (α₂γ₂; HbA) usually proceeds within six months after parturition, which unfortunately also proceeds on schedule in patients with hemoglobinopathies. However, in the majority of these individuals, the upstream y globin genes are intact and fully functional, and if these could be reactivated, functional hemoglobin synthesis could be maintained during adulthood, ameliorating the severity of the disease (Atweh et al., 2001). This is suggested by observations of the mild phenotype of individuals with co-inheritance of homozygous β-thalassemia and hereditary persistence of fetal hemoglobin (HPFH), and by those patients with homozygous β°-thalassemia who synthesize no adult hemoglobin, but in whom a reduced requirement for transfusions is observed in the presence of increased concentrations of fetal hemoglobin.

The first group of compounds discovered having HbF reactivation activity are cytotoxic drugs. 5-azacytidine was initially found, in experimental animals, impressing cellular control of fetal hemoglobin in the adult (DeSimone et al., 1982). Later baboons treated with cytotoxic doses of arabinosylcytosine (ara-C) responded with striking elevations of F-reticulocytes (Papayannopoulou et al., 1984). Induction of γ-globin also occurred in monkeys or baboons treated with hydroxyurea (Letvin et al., 1984). Vinblastine, an M stage-specific agent that arrests cells in mitosis, also produces perturbations of erythropoiesis and stimulates HbF synthesis in baboons (Veith et al., 1985). Following these studies, hydroxyurea was used for induction of HbF in humans and later became the first and only drugs approved by the Food and Drug Administration (FDA) for the treatment of hemoglobinopathies. However, the pharmacologic induction of HbF through the mechanism of late progenitor cell cytotoxicity seems to reach a dead end in drug discovery. It is unlikely that a cytotoxic compound better than hydroxyurea could be found.

The second group of compounds capable of HbF reactivation activity was short chain fatty acids. Initially, the seminal observation led to the discovery of γ-aminobutyric acid, which is acting as a fetal hemoglobin inducer (Perrine et al., 1987). Subsequent studies showed that butyrate stimulated globin production in adult baboons (Constantoulakis et al., 19888), and it induced γ globin in erythroid progenitors in adult animals or patients with sickle cell anemia (Perrine et al., 1989). Derivatives of short chain fatty acids such as phenylbutyrate (Dover et al., 1994) and valproic acid (Liakopoulou et al., 1995) also induce HbF in vivo. Since there exist a very large number of short chain fatty acid analogs or derivatives that are potential inducers of HbF, there are ample opportunities for discovering HbF inducers that are more potent than butyrate. Phenylacetic and phenylalkyl acids (Torkelson et al., 1996), which were discovered during subsequent studies, belonged to such examples. Presently, however, the use of butyrate or its analogs in sickle cell anemia and β-thalassemia remains experimental and cannot be recommended for treatment outside of clinical trials.

Clinical trials aimed at reactivation of fetal hemoglobin synthesis in sickle cell anemia and β-thalassemia have included short term and long term administration of 5-azacytidine, hydroxyurea, recombinant human erythropoietin, and butyric acid analogs, as well as combinations of these agents. However, varying drawbacks contraindicate the long term use of such agents or therapies. For example, although the hydroxyurea stimulates fetal hemoglobin production and clinically reduces sickling crisis, it is potentially limited by myelotoxicity and the risk of carcinogenesis. Potential long term carcinogenicity also exists in 5-azacytidine-based therapies. Feasible clinical treatments for these diseases remain scarce. Erythropoietin-based therapies have not proved consistent among a range of patient populations. The short half-lives of butyric acid in vivo have been viewed as a potential obstacle in clinical settings.

Still, notable efforts have been made in discovery and developing new drugs for the treatment of sickle cell anemia and β-thalassemia. In the past decades, some progress has also been made in the pharmacological management of sickle cell anemia and thalassemia.

WO 9,712,855 (Tung) describes butyrate prodrugs derived from lactic acid for increasing gamma globin and fetal hemoglobin in a patient. The compounds disclosed in the application are particularly effective in treating beta-hemoglobinopathies, including sickle cell syndromes and beta-thalassemia syndromes.

U.S. Pat. No. 6,372,213 (Um, et al.) provides a method of treatment of sickle cell anemia or thalassemia with protein C. The patent provides a needed therapy for potentially serious and debilitating disorders while avoiding complications such as bleeding tendency, toxicity and general side effects of currently available anti-coagulant agents.

U.S. Pat. No. 6,312,707 (Markov, et al.) describes fructose-1,6-diphosphate (FDP) has been shown, in double-blinded controlled clinical trials on patients with sickle cell anemia, to substantially reduce the pain suffered by such patients during the recurrent ischemic crises that are caused by red blood cell sickling.

U.S. Pat. No. 6,231,880 (Perrine) describes a number of compositions for pulsed administration to treat human blood disorders such as sickle cell anemia or thalassemia. The compositions contain chemical compounds that stimulate the expression of fetal hemoglobin and stimulate the proliferation of red blood cells, white blood cells and platelets in patients and ex vivo for reconstitution of hematopoiesis in vivo.

U.S. Pat. No. 6,028,103 (Brugnara, et al.) discloses that triaryl methane compounds or analogues are useful as efficacious drugs in the treatment of sickle cell disease and diseases characterized by unwanted or abnormal cell proliferation. The compounds inhibit mammalian cell proliferation, inhibit the Gardos channel of eryrocytes, reduce sickle erythrocyte dehydration and delay the occurrence of erythrocyte sickling or deformation.

U.S. Pat. No. 5,945,407 (Bemis, et al.) describes uses of butyrate esters of threitol in pharmaceutically increasing fetal hemoglobin and gamma globin in a patient, and particularly in treating beta-hemoglobinopathies, such as sickle cell syndromes and beta-thalassemia syndromes.

U.S. Pat. No. 5,753,632 (Schmidt, et al.) describes the use of colloidal silica for the treatment of sickle-cell anemia, malaria and exogenously induced leucopenias, which leads to a significant improvement in the condition of the patients.

U.S. Pat. No. 5,665,392 (Kumar, et al.) describes a pharmaceutical formulation useful for treating patients suffering from thalassemia, which comprises powder of Anemonin Pretensis in an amount in the range of 0.02 to 0.12 wt % of the formulation, quinine sulphate in an amount in the range of 0.0005 to 0.003 wt % of the formulation, distilled or demineralised water in an amount in the range of 0 to 40 wt % of the formulation and, ethanol in an amount in the range of 99.88 to 60 wt % of the formulation; and a process for preparing the formulation by mixing the above ingredients.

U.S. Pat. No. 5,447,720 (Fadulu) describes a composition extracted from alfalfa and other certain plant materials for the treatment of hemoglobinopathies. The plant material is first extracted with 1,1,1-trichloroethane and a hydroxide base, followed by extraction with hexane. The polar acidic compounds present in alfalfa and other plant materials selectively dissolve in the hexane phase and exhibit good antisickling activity in vitro. Further, these active compounds which comprise the inventive extract are effective in vivo by significantly alleviating the many clinical manifestations of sickle cell anemia and thalassemia patients.

U.S. Pat. No. 5,925,356 (Subbiah) provides a method of isolating and purifying cucurbitacins. The method involves the production of a cucurbitacin-containing liquid from the plant matter containing cucurbitacins. The liquid is then sequentially extracted with a non-polar solvent and then a moderately polar solvent. In a preferred embodiment, the cucurbitacin is purified by flash-column chromatography.

EP 0,627,220 (Hayhurst, et al.) describes pharmaceutical compositions containing butyric acid derivatives, particularly isobutyramide, are advantageously indicated for the therapy of thalassemia when compared with known formulations.

EP 0,617,966 (Perrine, et al.) describes a method for inhibiting the gamma-globin to beta-globin switching in subjects afflicted with beta-globin disorders. It ameliorates the clinical symptoms of sickle cell disease or beta-thalassemia by introducing activin or inhibin into the subject prior to natural completion of the switching process.

Although these efforts have led to novel advances in developing new drugs for the treatment of hemoglobinopathies, most of them are still under further investigation. The present treatment of the sickle cell anemia and P-thalassemia apparently is not satisfactory. Therefore, it is of importance to develop new pharmaceutics for effectively treating and/or managing various anemia conditions, including, but not limited to, the inherent varieties.

### SUMMARY OF THE INVENTION

An object of the present invention is to provide a method of making a medicament or pharmaceutical composition suitable for administering to human subject or other mammals for treating anemia conditions using a compound of the cucurbitacin analog or using an extract of an herbal medicine which contains a compound of the cucurbitacin analog. A preferred compound is cucuibitacin D. A preferred herbal medicine for this use is Trichosanthes.

Another object of the present invention is to provide a method of treating anemia conditions in a mammalian subject by administering to the subject a pharmaceutically effective amount of a compound of the cucurbitacin analog or using an extract of an herbal medicine which contains a compound of the cucurbitacin analog. For practicing this method, a preferred compound is cucurbitacin D. A preferred herbal medicine for this method is Trichosanthes.

Another object of the present invention is to provide a pharmaceutical composition comprising a pharmaceutically acceptable carrier and a pharmaceutically effective amount of a compound of the cucurbitacin analog or an extract of an herbal medicine which contains a compound of the cucurbitacin analog. For this pharmaceutical composition, a preferred compound is cucurbitacin D. A preferred herbal medicine for this composition is Trichosanthes. Such pharmaceutical composition is accompanied with an indication informing that the pharmaceutical composition is suitable for treating or improving one or more anemia conditions.

Another object of the present invention is to provide a method of inducing hemoglobin expression, and/or reactivating fetal and/or adult hemoglobin, and/or inducing γ-globin expressing in red blood cells by contacting the red blood cells with a compound of the cucurbitacin analog or an extract of an herbal medicine which contains a compound of the cucurbitacin analog. For practicing this method, a preferred compound is cucurbitacin D. A preferred herbal medicine for this method is Trichosanthes.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF DRAWINGS

FIG. 1 shows a particular extract from Trichosanthes prepared according to the present invention contains substantially a single compound.

FIG. 2 shows the single compound in FIG. 1 having a single sharp and symmetry peak with a retention time of 7.935 minutes in a HPLC profile.

FIG. 3 shows the dose-response curve of the single compound in FIG. 1 on hemoglobin induction in the K562 cell line.

FIG. 4 shows the dose-response curve of cucurbitacin D on hemoglobin induction in the K562 cell line. K562 cells were treated with different doses of cucurbitacin D and cultured in 96-well plate for 6 days.

FIG. 5 is the result of transcriptional analysis of α- and γ-globin gene by RT-PCR. K562 cells were cultured for 6 days and total RNA was prepared followed by RT-PCR analysis.

FIG. 6 is the result of flow cytometry analysis showing the fetal hemoglobin expression in K562 cells with different treatments (I: negative control; II: untreated; III: 25 mg/ml hydroxyurea and IV: 12.5 ng/ml cucurbitacin D).

FIG. 7 shows the hemoglobin positive cell percentage of human PBMC derived erythroid progenitor cells treated by different compounds, including cucurbitacin D of the present invention.

FIG. 8 shows immunofluorescence confocal microscopicimages of human BM mononuclear cells treated by different compounds, including cucurbitacin D of the present invention.

FIG. 9 shows the effect of cucurbitacin D and hydroxyurea on fetal hemoglobin expression in beta-thalassemia major patient progenitor cells.

### DETAILED DESCRIPTION OF THE INVENTION

Natural Resources for Obtaining Cucurbitacin Analogs

For the purpose of practicing the present invention, there are rich natural resources to obtain cucurbitacin analogs. Cucurbitacins belong to a group of complex compounds found in plants of the Cucurbitaceae family. The bitter taste in the eggplant or cucumber, members of the Cucurbitaceae family, are due to the presence of cucurbitacins. The cucurbitacin analogs commonly found in nature resources are cucurbitacins A, cucurbitacins B, cucurbitacins D, cucurbitacins E, cucurbitacins I and cucurbitacins Q. Cucurbitaceaes species that are rich in cucurbitacins, include Trichosanthe, Cucurbita pepo, Cucumis sativus and Citrullus ecirrhosus. Other herbs such as Picrorhiza kurroa of the Scrophulariaceae family (Stuppner and Wagner, 1989) and Iberis umbellate of the Brassicaceae family (Dinan, 1997) are also found rich in cucurbitacins. Cucurbitacins can be found in all parts of the plant, but usually are more concentrated in the seeds and fruits. Some members of Cucurbitaceae with high levels of cucurbitacins have been used as traditional herbal medicines for a long time. For example, Trichosanthes has been widely prescribed by herbal medical practitioners for thousands of years in China. Therefore, these herbal plants provide rich resources for obtaining various cucurbitacin analogs in practicing the present invention. On the other hand, synthetic effort can be made to provide alternative routes of obtaining pharmaceutically useful cucurbitacin analogs, which may be carried out on a larger scale and more economically.

The following lists the chemical structures, formula and mass of cucurbitacin analogs, including cucurbitacin A, B, C, D, E, F, H, I, J, L, O, P, Q and S:

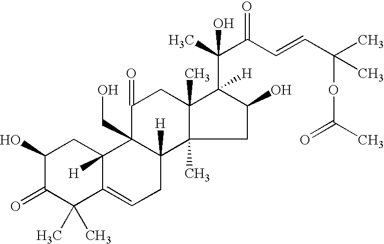

cucurbitacin A
Formula C₃₂H₄₆O₉
Mass 574.314

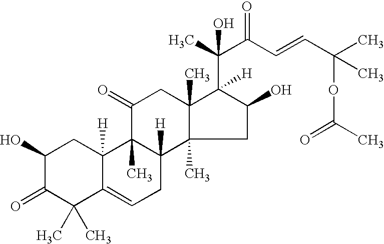

cucurbitacin B
Formula C₃₂H₄₆O₈
Mass 558.3192

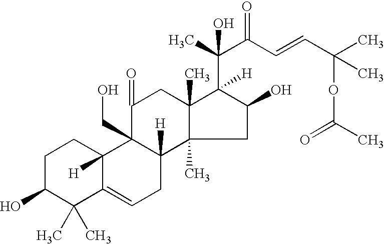

cucurbitacin C
Formula C₃₂H₄₈O₈
Mass 560.3348

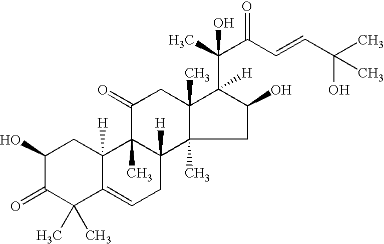

cucurbitacin D
Formula C₃₀H₄₄O₇
Mass 516.3087

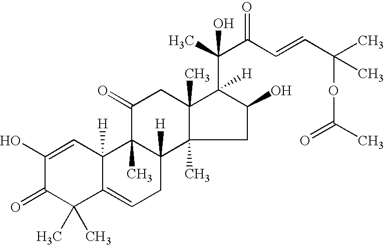

cucurbitacin E
Formula C₃₂H₄₄O₈
Mass 556.3035

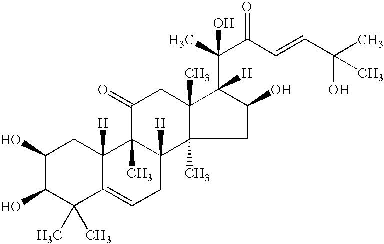

cucurbitacin F
Formula C₃₀H₄₆O₇
Mass 518.3243

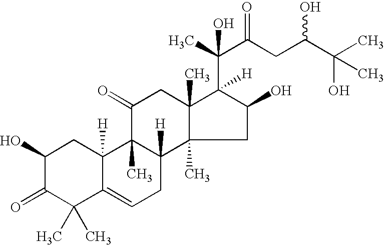

cucurbitacin H
Formula C₃₀H₄₆O₈
Mass 534.3192

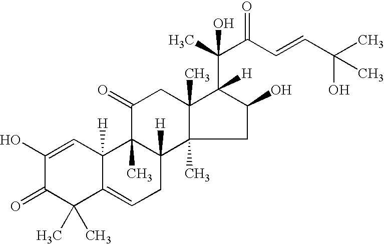

cucurbitacin I
Formula C₃₀H₄₂O₇
Mass 514.293

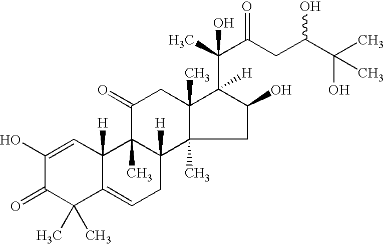

cucurbitacin J
Formula C₃₀H₄₄O₈
Mass 532.3036

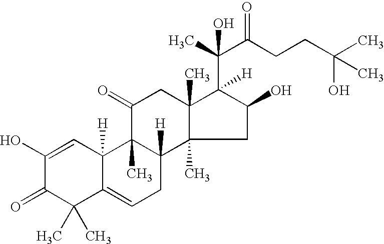

cucurbitacin L
Formula C₃₀H₄₄O₇
Mass 516.3087

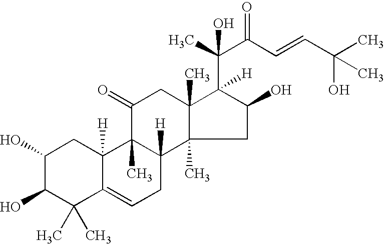

cucurbitacin O
Formula C₃₀H₄₆O₇
Mass 518.3243

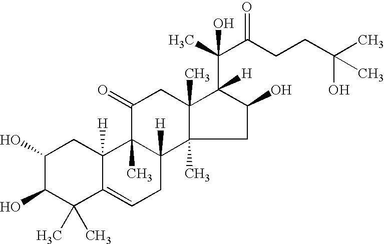

cucurbitacin P
Formula C₃₀H₄₈O₇
Mass 520.3399

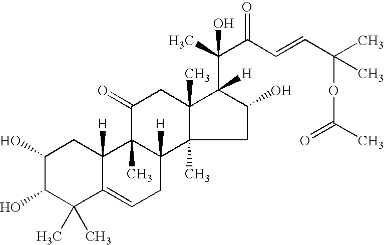

cucurbitacin Q
Formula C₃₂H₄₈O₈
Mass 560.3348

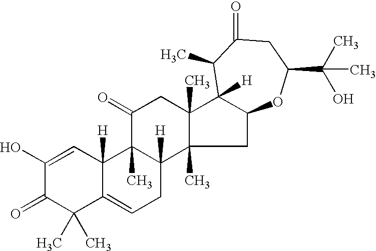

cucurbitacin S
Formula C₃₀H₄₂O₆
Mass 498.298

For the purpose of understanding the present invention and construing the scope of the appended patent claims, “cucurbitacin analog” means any compound having a backbone structure shared by the above listed examples. It is contemplated that a cucurbitacin analog shares the similar properties and pharmaceutical applications as cucurbitacin D in practicing the present invention. Cucurbitacin D is an exemplified embodiment disclosed herein. It is also contemplated that a cucurbitacin analog, such as cucurbitacin D, may be obtained through total synthesis or semi-synthesis.

It is further contemplated, as a person with ordinary skill in the art would understand, that a cucurbitacin analog, such as cucurbitacin D, may be made in various possible racemic, enantiomeric or diastereoisomeric isomer forms, may form salts with mineral and organic acids, and may also form derivatives such as N-oxides, prodrugs, bioisosteres. “Prodrug” means an inactive form of the compound due to the attachment of one or more specialized protective groups used in a transient manner to alter or to eliminate undesirable properties in the parent molecule, which is metabolized or converted into the active compound inside the body (in vivo) once administered. “bioisostere” means a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Making suitable prodrugs, bioisosteres, N-oxides, pharmaceutically acceptable salts or various isomers from a known compound (such as those disclosed in this specification) are within the ordinary skill of the art. Therefore, the present invention contemplates all suitable isomer forms, salts and derivatives of the above disclosed cucurbitacin analogs, which are all within the meaning of “cucurbitacin analog” for the purpose of construing the claims.

Making Herbal Extracts and Isolating Cucurbitacins Therefrom

The extraction of herbal plant Cucurbitaceaes may be performed according to any conventional methods known in the art. The following flow-chart presents an exemplary extracting process, which can be employed to make extracts from a plant of the Cucurbitaceaes family, for example, Trichosanthe.

According to the flow-chart, the original plant materials may be sliced, dried, or physically disintegrated prior to processing. Then the extraction is preferably conducted by soaking the dried plant tissues in water or polar organic solvents or their mixture at any ratio. Such mixture should be enclosed and incubated at a certain temperature, which is usually, but not limited to, ranges between the room temperature and boiling temperature of the solvent. Resulting extract contains biological active ingredients and compounds in liquid phase. The liquid phase is isolated from the remaining insoluble materials by any means known in the art, but preferably by filtrating through medical gauze. Remaining insoluble materials may be further removed by centrifugation. The resulting liquid (Fraction A) is typically clear and additional filtration will be performed if necessary. The previous obtained Fraction A can be optionally further concentrated into a viscous liquid phase by any means known in the art, preferably by rotary evaporation. Fraction A can also be optionally extracted with a non-polar solvent to remove those essentially produced contaminants as pigments, lipids, fatty acids and waxes from aqueous phase.

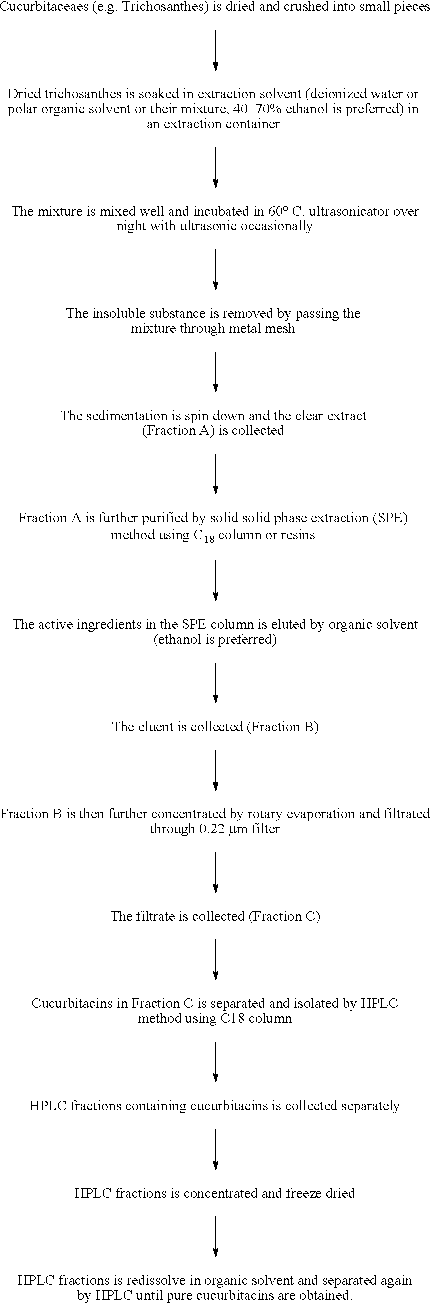

Further purified ingredients can be obtained if Fraction A is processed by subsequent separation methods. Examples of such methods include, but not limited to, liquid-liquid extraction, solid phase extraction (SPE), super filtration, super critical extraction, etc. For liquid-liquid extraction, a polar organic solvent is always provided to extract a mixture of partially purified ingredients. For SPE, the column is generally eluted by a first polar organic solvent to remove the irrelative ingredients, and then eluted by a second polar organic solvent, usually with less polarity index, to wash out ingredient comprising the active compounds. Finally the second elution solvent is collected (Fraction B). This Fraction B is then further concentrated by rotary evaporation and filtrated through 0.22 μm filter (Fraction C).

Single compound responsible for the biological activity can be isolated from Fraction C by further separation methods. Examples of such methods include, but not limited to, thin layer chromatography (TLC), gas chromatography (GC), liquid chromatography (LC), and high-performance liquid chromatography (HPLC), of which HPLC is preferred. Different columns can be adopted during HPLC purification. Examples of such columns include, but not limited to, normal phase columns, reverse phase columns, ion-exchange columns, and size-exclusion columns, of which C18 reverse phase columns are preferred.

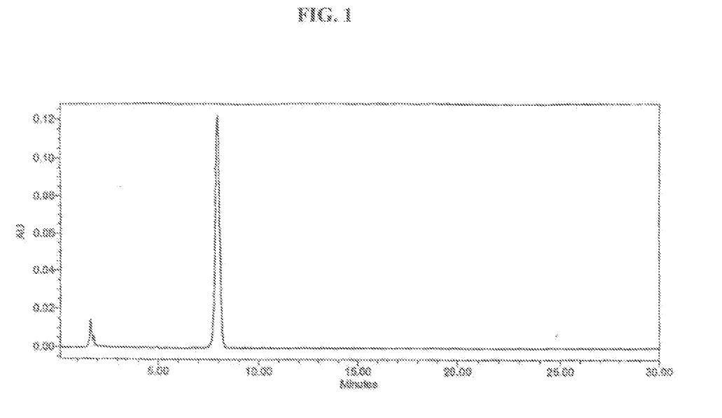

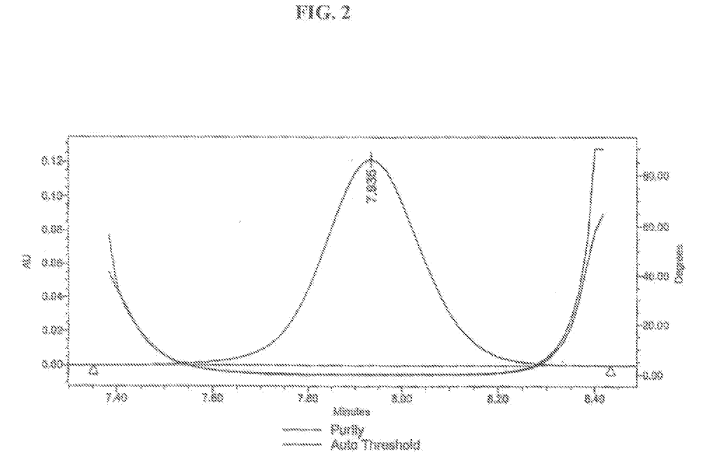

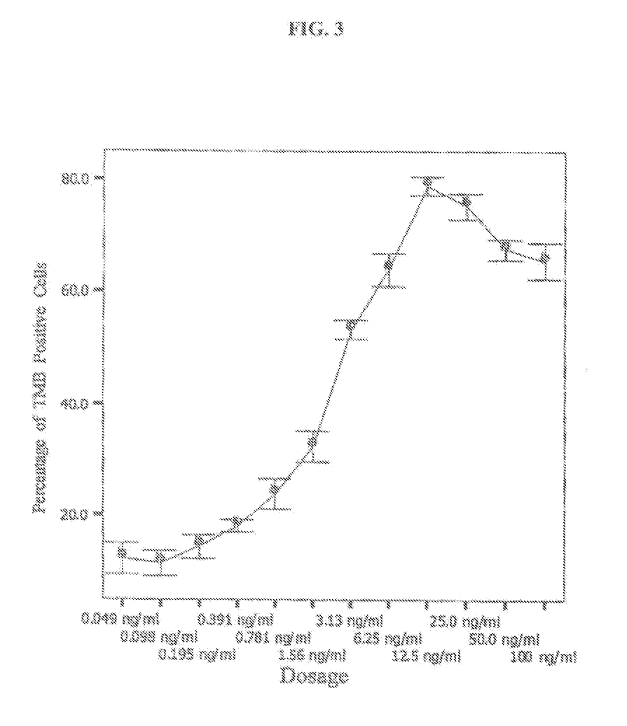

In one embodiment of the invention, the active compound is purified by reverse phase C18 HPLC, using a gradient elution protocol, from 0% to 60% methanol. The resulting product will comprise active compound in essentially pure form. However, the purity can be improved if the HPLC purification process is repeated. FIG. 1 provides an example of the pure active compound (referred to as “Lead compound”) derived from HPLC separation, the purity plot (from Waters® Millenmium32 software) of which is indicated in FIG. 2. FIG. 3 provides an example of the ultraviolet/visible spectrum of the pure active compound derived from the photodiode array (PDA) detector from HPLC separation module (Waters® alliance® 2695/PDA 996). To get the powder form of the compound, the collected solution should be rotary evaporated followed by frozen and lyophilized. This active compound is identified to be cucurbitacin D.

Cucurbitacins in a deacetylation form, a form which is naturally occurring in very trace amount or even not occurring, may be more biologically active than the parent compounds. Deacetylation of acetyl group(s) to produce a corresponding deacetylated cucurbitacin analogue (which is also referred to as “deacetylated entity”) can be effected by various chemical reactions. Examples of such reactions include, but not limited to, saponifaction by potassium carbonate, sodium methoxide and magnesium methoxide in methanol, as well as reduction by lithium aluminum hydride and other reducing agents, of which potassium carbonate in methanol is preferred. A range of cucurbitacins can be subject to this deacetylation reaction. Below is an example, showing cucurbitacin B undergoing deacetylation to afford cucurbitacin D:

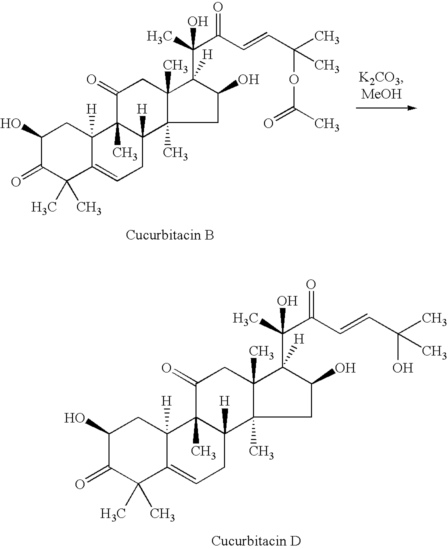

### EXAMPLE 1

### Laboratory-Scale Preparation of Cucurbitacin B and Cucurbitacin D

A) Crude Extract

One kilogram of cucurbitacin-containing plant, Trichosanthes, is crushed into small pieces and oven dried. 40% ethanol is added into the Trichosanthes for extraction in a 5 L bottle (ratio approximately: 1 kg herb: 4 L extraction solvent). The mixture is mixed well and incubated in a 60° C. ultrasonicator over night with sonication occasionally. Then the insoluble substance is removed by passing the mixture through a cheese cloth. Then the sedimentation is spun down and clear fitrate is collected.

B) Solid Phase Purification

The extract, i.e., the clear filtrate from step A, is further purified by solid phase extraction method using C18 column. The extract is firstly loaded into the absorbent C18 and the cucurbitacins are eluted by ethanol. The cucurbitacin-containing eluent is collected in sample collection tube. The eluent is then rotary evaporated to a small volume. Ethanol is added into the eluent until a clear solution obtained.

C) First HPLC Purification

The herbal extract from step B is then purified by HPLC technique using C₁₈ column. It is firstly purified by a Waters© Atlantis d C₁₈ column (10 mm×150 mm) using 60% acetonitrile and 40% water as mobile phase. The fraction containing cucurbitacin B and cucurbitacin D is collected.

D1) Purification of Cucurbitacin B

The fraction containing cucurbitacin B from step C is then purified by Waters© Symmetry Prep C₁₈ column (7.8 mm×150 mm) using 60% methanol and 40% water as mobile phase and the fraction containing cucurbitacin B is collected. The collected fraction is then purified again by Waters© Symmetry Prep C₁₈ column (7.8 mm×150 mm) using 40% acetonitrile and 60% water as mobile phase to obtain pure cucurbitacin B.

D2) Purification of Cucurbitacin D

The fraction containing cucurbitacin D from step C is then purified by Waters© Symmetry Prep C₁₈ column (7.8 mm×150 mm) using 55% methanol and 45% water as mobile phase and the fraction containing cucurbitacin D is collected. The collected fraction is then purified again by Waters© Symmetry Prep C₁₈ column (7.8 mm×150 mm) using 28% acetonitrile and 72% water as mobile phase and the fraction containing cucurbitacin D is collected. The collected faction is finally purified by a Waters© Atlantis d C₁₈ column (10 mm×150 mm) using 60% methanol and 40% water as mobile phase to obtain pure cucurbitacin D.

### EXAMPLE 2

Large-Scale Preparation of Cucurbitacin B and Cucurbitacin D

A) Crude Extract

Twenty kilograms of cucurbitacin-containing plant, Trichosanthes, are crushed into small pieces and oven dried. 40% ethanol is added into the Trichosanthes for extraction in a 100 L reaction tank (ratio approximately: 1 kg herb: 4 L extraction solvent). The mixture is mixed well and incubated in a 60° C. with constant stirring. The insoluble substance is removed by passing the mixture through a metallic mesh. Then the extract is allowed to settle at room temperature for overnight and the upper clear solution is obtained.

B) Solid Phase Purification

The extract is passed through a large column packed with DM11, an absorbent, and cucurbitacins adhered on the resins are eluted by ethanol. The eluent is concentrated and adjust to ethanol content below or equal to 40%. It is then purified by solid phase extraction method using C18 column. The extract is loaded into the absorbent (DM11) and cucurbitacins are eluted by ethanol. The cucurbitacin-containing elutent is collected in a sample collection vessel. The eluent is then rotary evaporated to a smaller volume. Ethanol is added into the eluent until a clear solution obtained.

C) First HPLC Purification

The herbs extract from section B is then purified by preparative HPLC technique using C18 columns. It is firstly purified by a Waters© Xterra RP₁₈ column (19 mm×150 mm) using ethanol and water as mobile phase running in gradient, where ethanol content from 35% to 50%. The fraction containing cucurbitacin B and cucurbitacin D is collected.

D1) Purification of Cucurbitacin B

The fraction containing cucurbitacin B from step C is then purified by Waters© Symmetry Prep C₁₈ column (19 mm×150 mm) using 45% ethanol and 55% water as mobile phase and the fraction containing cucurbitacin B is collected. The collected fraction is then purified again by Waters© SunFire Prep OBD C₁₈ column (19 mm×150 mm) using 40% acetonitrile and 60% water as mobile phase to obtain pure cucurbitacin B.

D2) Purification of Cucurbitacin D

The fraction containing cucurbitacin D from step C is then purified by Waters© Symmetry Prep C₁₈ column (19 mm×150 mm) using 40% ethanol and 60% water as mobile phase and the faction containing cucurbitacin D is collected. The collected fraction is then purified again by Waters© SunFire Prep C₁₈ column (19 mm×150 mmn) using 35% acetonitrile and 65% water as mobile phase and the fraction containing cucurbitacin D is collected. The collected fraction is finally purified by a Waters© XBridge Prep C₁₈ column (10×150 mm) 30% acetonitrile and 70% water as mobile phase to obtain pure cucurbitacin D.

### EXAMPLE 3

Conversion of Cucurbitacin B to Cucurbitacin by Deacetylation

15 mg of cucurbitacin B from Example 1(D1) or Example 2 (D1) is added to a mixture containing excess amount of potassium carbonate (Aldrich) in dry methanol. The mixture is stirred under nitrogen or argon at room temperature for 3 hours and is quenched with excess amount of saturated ammonium chloride (Aldrich). The aqueous mixture is then extracted with ethyl acetate twice. The salt in the combined organic extract is removed by passing through a short pad of silica gel (Merck) and eluted with ethyl acetate. The solvent is removed by rotary evaporation and the resultant crude oil is suspended in methanol for separation according to the method in Example 1(D1) or Example 2 (D1).

### EXAMPLE 4

Extracts, A, B and C were obtained by a procedure detailed in the following:

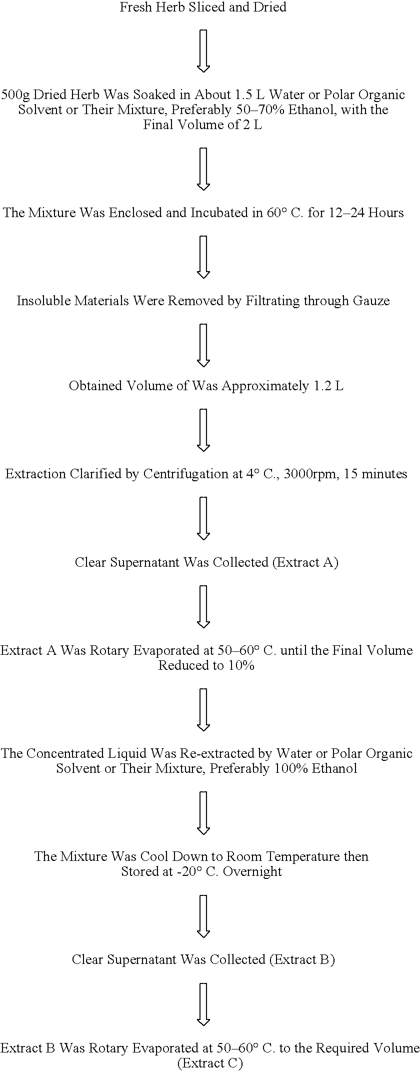

### EXAMPLE 5

Ingredients, A, B and C were further isolated from Extract B of Example 4 by a procedure detailed in the following:

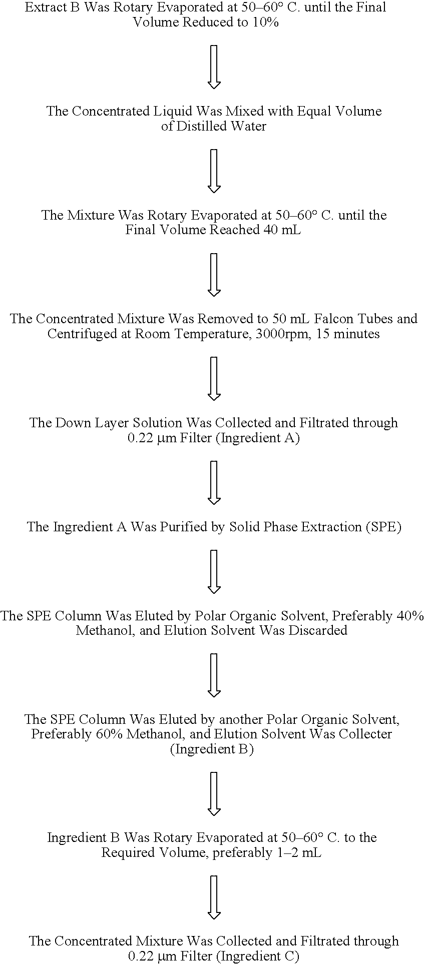

For the purpose of construing the claims, the term “extract” includes, but is not limited to, Extract A, Extract B, Extract C, Ingredient A, Ingredient B and Ingredient C. The present invention completes any form of extracts made according to any methods known to people ordinarily skilled in the art.

Biological Effects of the Herbal Extract and Isolated Cucurbitacin

(A) Cucurbitacin D Induced Hemoglobin Expression on K562 Cells

The K562 cell line is considered to be a multipotent hematopoietic stem cell because it has multiple-lineage markers. The cells could be induced to erythrocytic, monocytic, granulocytic and megakariocytic differentiation using various materials. Since its discovery, K562 cell line has been extensively used as a model in studies of erytlroid differentiation and regulation of globin gene expression. K562 cells (ATCC) were cultured in RPMI 1640 (Gibco) supplemented with 10% fetal calf serum (FCS, Gibco) and 1% PSN (Gibco). The cultures were maintained under a humidified atmosphere with 95% air/5% CO₂ at 37° C. The hemoglobin positive K562 cells can be identified by 3,3′,5,5′-tetramethylbenzidine (1 MB, Sigma) staining. In brief, 10 μl cell suspension was pipetted out and mixed with 10 μl TMB working solution. Five minutes later, cells were scored as positive (blue) or negative (pale yellow) at 200× microscope. Before the assay, K562 cells were scored first by TMB staining to ensure there was no self differentiation (TMB positive cell percentage less than 1.0%). 100 μl/well complete RPMI 1640 was added into a 96-well plate, and 180 μl was added in the first well. 20 μl Trichosanthes extract (or compounds to be tested) was added into the first well immediately. Then 100 μl of the mixture from the previous well (the first well) was transferred to the next well (the second well). In the same fashion, 100 μl was transferred from the second to the third. This dilution process is continued until the last well so that each well on the plate contains half of the Trichosanthes extract amount as contained in the previous well but twice as mush as the next well. 100 μl K562 cell suspension with the density of 4×10⁴ cells/ml was then added to each well of the 96-well plate. The final cell suspension was mixed well and cultured in the 37° C. CO₂ incubator for six days. Then the hemoglobin positive cells were recorded by TMB staining. In addition to various Trichosanthes extracts (Extracts A, B, C; Ingredients A, B, C), purified cucurbitacin D and some previously reported compounds with potential activity on hemoglobin induction were also tested in the same fashion as above described. The results are presented in Table 1 and 2, which demonstrated that cucurbitacin D isolated from Trichosanthes, as well as various Trichosanthes extracts (Extacts A, B, and C) and ingredients (Ingredients A, B and C), can significantly induce erythrocytic differentiation and hemoglobin expression. Extacts A, B, and C were prepared according to Example 4 and Ingredients A, B and C were prepared according to Example 5. Preparation of Lead compound was described previously and shown in FIG. 1.

TABLE 1

| | TMB | | TMB |
|---|---|---|---|
| | Positive Cell | | Positive Cell |
| Components | Percentage | Components | Percentage |
| Negative Control | 10.3 ± 1.5 | Ingredient A | 63.1 ± 3.7 |
| Hydroxyurea | 66.7 ± 4.4 | Ingredient B | 61.9 ± 4.8 |
| Extract A | 56.8 ± 3.9 | Ingredient C | 66.5 ± 3.6 |
| Extract B | 58.6 ± 5.1 | Lead Compound | 68.7 ± 4.1 |
| Extract C | 58.1 ± 4.4 | | |

Note:
The dosage of hydroxyurea (positive control) is 25.0 μg/ml.
The TMB positive percentage refers to the group of cells that treated with optimal dosage of Trichosanthes extraction components and showed maximal positive cell percentage.

TABLE 2

| | Amifostine | 5-AzaC | rhEPO | Hydroxyurea | SPB | cucurbitacin D |
|---|---|---|---|---|---|---|
| Dilution | (200 μg/ml) | (10 μg/ml) | (20 U/ml) | (100 μg/ml) | (100 μg/ml) | (100 ng/ml) |
| 1 | / | 17.3 ± 1.5 | 13.2 ± 0.9 | / | 3.40 ± 0.3 | 49.5 ± 2.1 |
| ½ | / | 22.1 ± 1.7 | 14.1 ± 1.0 | / | 8.9 ± 0.4 | 51.6 ± 2.5 |
| ¼ | / | 25.4 ± 1.7 | 15.6 ± 1.2 | 69.5 ± 3.8 | 10.6 ± 0.9 | 53.7 ± 2.3 |
| ⅛ | 7.5 ± 0.7 | 35.9 ± 2.2 | 15.5 ± 0.8 | 41.7 ± 3.2 | 14.5 ± 1.2 | 66.8 ± 3.0 |
| 1/16 | 29.1 ± 1.9 | 26.7 ± 1.9 | 13.2 ± 0.5 | 22.3 ± 1.8 | 17.1 ± 1.7 | 54.2 ± 2.4 |
| 1/32 | 21.4 ± 1.8 | 14.5 ± 1.3 | 11.4 ± 1.1 | 14.4 ± 1.1 | 23.4 ± 1.8 | 39.8 ± 1.7 |
| 1/64 | 15.1 ± 1.1 | 11.2 ± 1.1 | 12.7 ± 0.7 | 11.5 ± 0.8 | 16.2 ± 1.2 | 22.5 ± 1.2 |
| 1/128 | 13.0 ± 1.2 | 12.3 ± 0.8 | 13.5 ± 1.2 | 12.5 ± 1.2 | 11.7 ± 1.3 | 16.7 ± 1.1 |
| 1/256 | 10.5 ± 0.6 | 10.9 ± 1.0 | 12.6 ± 1.1 | 12.6 ± 1.3 | 10.5 ± 0.7 | 12.5 ± 1.2 |
| 1/512 | 11.2 ± 0.8 | 10.8 ± 0.5 | 10.9 ± 0.6 | 11.4 ± 1.0 | 12.1 ± 0.9 | 12.2 ± 1.0 |
| 1/1024 | 10.9 ± 0.9 | 13.1 ± 0.7 | 12.1 ± 0.7 | 12.5 ± 1.4 | 12.9 ± 1.1 | 13.7 ± 0.8 |
| 1/2048 | 12.1 ± 1.1 | 11.6 ± 0.8 | 11.3 ± 0.8 | 13.3 ± 1.1 | 11.7 ± 1.1 | 11.4 ± 1.1 |
| Control | 11.0 ± 0.9 | | | | | |

(B) Dose-Dependent Effect of Cucurbitacin D

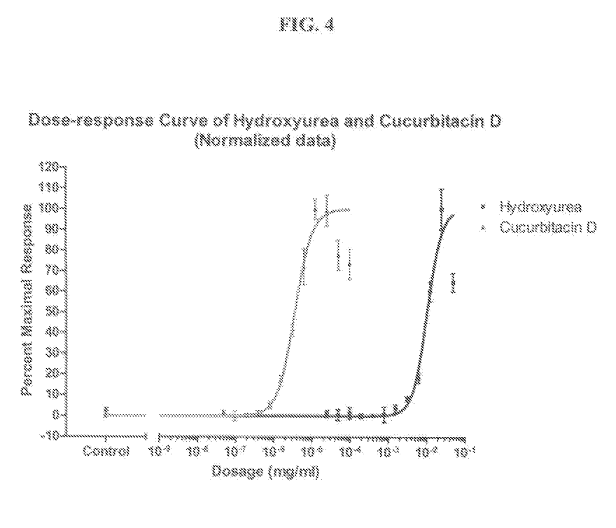

Dose-dependent effect of cucurbitacin D was studied on K562 cells which were cultured and assayed as described the previous section. Briefly, K562 cell suspension was mixed with serial two-fold diluted medium containing cucurbitacin D and cultured for six days. Then the hemoglobin positive cells were recorded by TMB staining. Similarly, K562 cells treated by a serial of two-fold diluted hydroxyurea or cucurbitacin D were cultured for 6 consecutive days. The concentration of cucurbitacin D used was serial diluted for 12 times, ranged from 100 ng/ml to 48.8 pg/ml, while hydroxyurea was from 50.0 μg/ml to 24.4 ng/ml. As shown in FIG. 4, the effect was dose-related and no effect was detected at a dosage lower than 0.1 ng/ml. However, a reduced effect was observed at the high dosage, which could be due to the cytotoxic effect of cucurbitacin D. This cytotoxic effect may be useful in antitumor applications. The dose-response curve provided the direct evidence that the hemoglobin inducing activity is attributed to cucurbitacin D. The results in FIG. 4 are normalized and indicated as mean±SD. (SD=standard deviation).

(C) Transcription Analysis of α- and γ-Globin Gene by RT-PCR

The mRNA of α- and γ-globin in cucurbitacin D and hydroxyurea treated K562 cells were analyzed by RT-PCR to investigate the globin gene expression at molecular level. The procedures for isolating total RNA were described as follows. The frozen cell lysate was thawed by incubating in a 70° C. water bath for 10 minutes with constant vortexing to shear the DNA. The thawed cell lysate was then chilled on ice immediately. The GT lysate was homogenized by drawing it into a sterile hypodermic syringe and expelling it through a 23-gauge needle for several times. Approximately 2 ml of the GT lysate was overlayered on 1 ml 5.7 M cesium chloride cushion in an ultra-centrifuge polyallomer tube. To avoid cracldng, the tubes were topped up with GT solution. The centrifuge tubes were balanced by adding in GT solution (with a deviation less than 0.01 g). It was then ultra-centrifuged at 32000 rpm for 18 hours at 18° C. After centrifugation, the overlaying supernatant was removed by aspiration. The tubes were inverted quickly and drained for a while to remove excess supernatant and to allow the RNA pellet to dry. Afterwards, the bottom 0.5 cm of the tube containing the clear RNA pellet was cut off with a new sterile scalpel blade. The RNA pellet was then rinsed out and resuspended very carefully to a new eppendorf tube with a total of 400 μl ddH₂O. An aliquot of 1 ml absolute ethanol and 45 μl of 3M sodium acetate (pH 4.8) were added. The RNA was pelleted by centrifugating at top speed for 30 minutes. The RNA pellet was washed with 70% ethanol to remove any residual salts. The vacuum dried RNA pellets were resuspended with ddH₂O to the final concentration of 0.1 μ/μl and followed by RT-PCR.

An aliquot of 10 μl diluted RNA solution was added to a new eppendorf tube and incubated in 65° C. water bath for 10 min to denature RNA. The RNA solution was chilled on ice for 2 min, followed by vortex and spin down. The following reaction agents were added respectively as following: M-MLV RT Buffer (5×, 4 μl), DTT (2 μl), RNAsin (40 U/μl, 1 μl), oligo dT (0.1 μg/μl, 1 μl), dNTP (10 mM, 1 μl), M-MLV Reverse Transcriptase (1 μl), RNA (0.1 μg/μl, 10 μl). The mixture was incubated at 37° C. for 1 hour. The reverse transcripted product was dilute by 80 μl H₂O to the final RNA concentration of 0.01 μg/μl. The standard PCR procedure was followed using 10 μl of diluted reverse transcripted product as template DNA each time. The PCR conditions for different primers were shown in the following Table 3.

TABLE 3

| | PCR Program | | | |
|---|---|---|---|---|
| | | Denaturing | | |
| Primer | Cycle | Temp. | Annealing Temp. | Extension Temp. |
| GAPDH | 18 | 94° C. | 56° C. | 72° C. |
| α-globin | 18 | 94° C. | 56° C. | 72° C. |
| γ-globin | 19 | 94° C. | 58° C. | 72° C. |

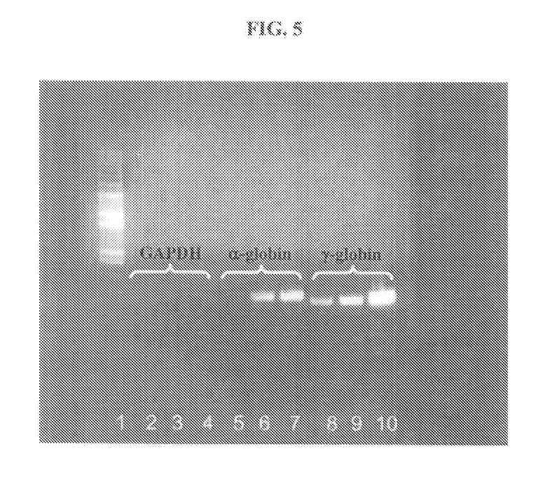

K562 cells treated with cucurbitacin D (12.5 ng/ml) and hydroxyurea (25.0 μg/ml) were cultured for six days. Total mRNA extracted was followed by RT-PCR analysis. As illustrated in FIG. 5, α- and γ-globin mRNA were increased in both hydroxyurea-treated and cucurbitacin D-treated cells, but more significantly in the latter. The results indicate that the fetal hemoglobin inducting activity of hydroxyurea and cucurbitacin D are likely to be caused by an increase in transcription of globin genes. The number in FIG. 5 represents as follows: Lane 1: Ikb plus DNA Marker; Lane 2: Control Cells, GAPDH Gene; Lane 3: Hydroxyurea-treated Cells, GAPDH Gene; Lane 4: Cucurbitacin D-treated Cells, GAPDH Gene; Lane 5: Control Cells, α-Globin Gene; Lane 6: Hydroxyurea-treated Cells, α-Globin Gene; Lane 7: Cucurbitacin D-treated Cells, α-Globin Gene; Lane 8: Control Cells, γ-Globin Gene; Lane 9: Hydroxyurea-treated Cells, γ-Globin Gene; Lane 10: Cucurbitacin D-treated Cells, γ-Globin Gene.

(D) FACS Analysis of K562 Cells Treated with Different Compounds

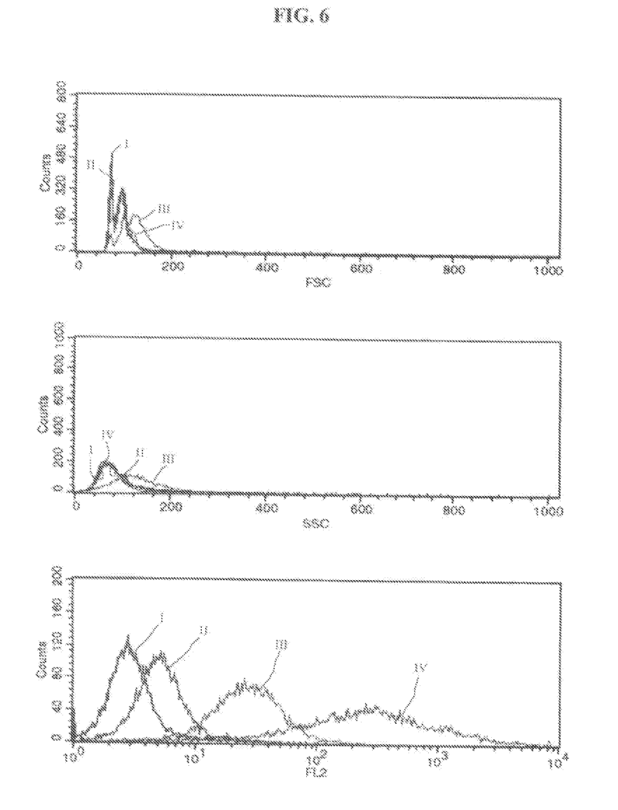

K562 cells were cultured with hydroxyurea (HU, 25 mg/ml) and cucrbitacin D (12.5 ng/ml) for 6 days. Then the cells were conjugated with PE labeled mouse anti human fetal hemoglobin monoclonal antibodies (Becton Dickinson) followed by FACS analysis. In brief, 1×10⁶ cells were collected in a 15 ml falcon tube and centrifuged in 150 rpm for 5 min. The cell pallet was washed with PBS. Then cells were fixed by gently mixing with 1 ml PBS with 4% formaldehyde (37-40%, Merck) for 1 hour at room temperature. The fixed cells were washed once with PBS and then were resuspended in 100 μl 0.01% Triton X-100 (Merck) in PBS/0.1% BSA (Sigma). 20 μl monoclonal antibody was added in, followed by mixing thoroughly and incubating for 30 min. in darkness at room temperature, with frequent gentle shaking. Finally the cells were washed with 1×PBS with 0.1% sodium azide once and kept in 4° C. until analysis. As shown in FIG. 6 from FSC and SSC, K562 cells treated by cucurbitacin D showed no obvious cell morphology changes, comparing with negative control and untreated cells. However, there existed obvious morphology changes in HU-treated cells. This could be due to the cytotoxicity of hydroxyurea in such high dosage. Expression of fetal hemoglobin was induced in both compound treated cells, but more significantly in the cucurbitacin D treated cells. The results also provide evidence that cucurbitacin D can positively induce not only adult hemoglobin, but also fetal hemoglobin expression. In FIG. 6, I is negative control; II is untreated; III is treated with 25 mg/ml hydroxyurea; and IV is treated with 12.5 ng/ml cucurbitacin D.

(E) Hemoglobin Expression on Peripheral blood monocytes (PBMC) Derived Human Erythroid Progenitor Cells Treated by Different Compounds

Peripheral blood was freshly phlebotomized and kept in heparinized tubes. PBMC were immediately isolated as described below. Freshly drawn human peripheral blood or bone marrow or mobilized peripheral blood, not older than 8 hours, was treated with an anti-coagulant (e.g. heparin, EDTA, citrate, acid citrate dextrose anticoagulant (ACD-A) or citrate phosphate dextrose (CPD)). The cells were diluted with 2-4 volumes of PBS. 35 ml of diluted cell suspension was carefully layered over 15 ml Ficoll Paque® (1.077 density) in a 50 ml conical tube and centrifuged at 400×g for 30-40 minutes at 20° C. in a swinging-bucket rotor (without brake). The upper layer was aspirated, leaving the mononuclear cell layer undisturbed at the interphase. The interphase cells (lymphocytes, monocytes, and thrombocytes) were carefully transferred to a new 50 ml conical tube. The conical tube was filled with PBS, mixed and centrifuged at 300×g for 10 minutes at 20° C. The supernatant was carefully removed completely. For removal of platelets, the cell pellet was resuspended in 50 ml of buffer and centrifuge at 200×g for 10-15 minutes at 20° C. The supernatant was carefully removed completely. By repeating this last washing step, most of the platelets remained in the supernatant upon centrifuigation at 200×g. Alternatively, the cells suspended in PBS or medium were layer on NycoPrep™ (1.063 density) and centrifuge for 15 minutes at 350×g. The mononuclear cells precipitated and the platelets remained in the supernatant. The cell pellet was resuspended in an appropriate buffer. The cells were counted and preceded to two phase culture system.

Epo-independent Phase I: Isolated peripheral blood (PB) cultured at a density of 5×10⁶ cells/ml in RPMI 1640 medium supplemented with 10% FCS and 1% PSN and 10% human bladder carcinoma cell line 5637 conditioned medium (CM5637). The CM5637 were prepared as following: human bladder carcinoma cells 5637 were cultured for 10 days. The medium was collected and centrifuged at 300×g for 10 minutes. The supernatant was filter sterilized and stored at 4° C. until use. The cultures were maintained under a humidified atmosphere with 95% air/5% CO₂ at 37° C. for 5 days.

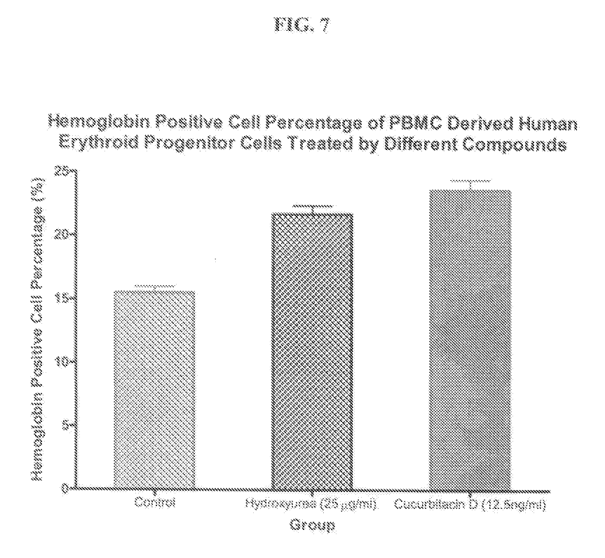

Epodependent Phase II: The nonadherent cells were harvested and washed by PBS after 5 days culture in phase I. The nonadherent cells were cultured in freshly prepared medium for 4 days. The fresh medium composed of 30% FCS, 1% BSA, 1×10⁻⁵ mol/L β-mercaptoethanol, 1.5 mmol/L glutamine, 1×10⁻⁶ mol/L dexamethasone, and 1 U/mL rhEPO in RPMI 1640. After cultured for 4 days in phase II, the lymphocytes were removed as described. The cells were spun down, harvested and resuspended in PBS. The medium was saved. The cells in PBS were carefully layered on a Percoll® solution (1.0585 density) in a 50 ml conical tube and centrifuged at 1,000×g for 20 minutes at room temperature. The upper layer solution, which containing the proerythroblasts, was collected and transferred to a new 50 ml conical tube. The conical tube was filled with PBS, mixed and centrifuged at 300×g for 10 minutes at 20° C. The supernatant was carefully removed completely. The cells were resuspended in the saved medium supplemented with hydroxyurea (25.0 μg/ml) or cucurbitacin D (12.5 ng/ml) and cultured under a humidified atmosphere with 95% air/5% CO₂ at 37° C. for 7 days before further analysis. Human PBMC/BM/PBSC derived progenitor cells were cultured in a density of 5×10⁶ cells/ml in RPMI1640 medium supplemented with 30% fetal calf serum (FCS), 1% PSN, 10% CM5637, and 1 U/ml rhEPO. For assay groups, hydroxyurea (25.0 μg/ml) or cucurbitacin D (12.5 ng/ml) was also added in the medium. The cultures were maintained under a humidified atmosphere with 95% air/5% CO₂ at 37° C. Seven days after the addition of hydroxyurea and cucurbitacin D, the cells were assayed by TMB staining as described in example 5 and the number of hemoglobin positive cells were scored. The result indicates both HU and cucurbitacin D were able to induce hemoglobin expression in normal erythroid progenitor cells. The activity of cucurbitacin D appeared to be better than hydroxyurea (FIG. 7).

(F) Immunofluorescence Confocal Microscope Analysis of Fetal Hemoglobin Expression

With the newly available anti-fetal hemoglobin monoclonal antibodies, it is convenient to detect the fetal hemoglobin containing cells (F cells) by confocal microscopy. Fetal hemoglobin belongs to intracellular antigens which need to be detected by membrane permeablizing techniques. The fetal hemoglobin expression of human BM mononuclear cells and K562 cells treated with cucurbitacin D and hydroxyurea were analyzed by immunofluorescence confocal microscope.

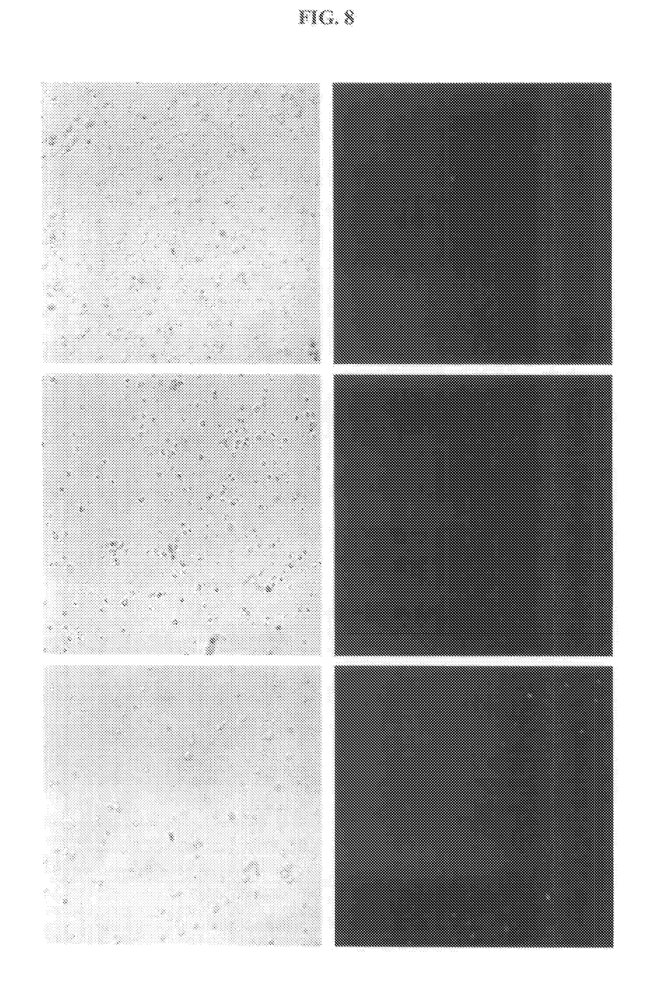

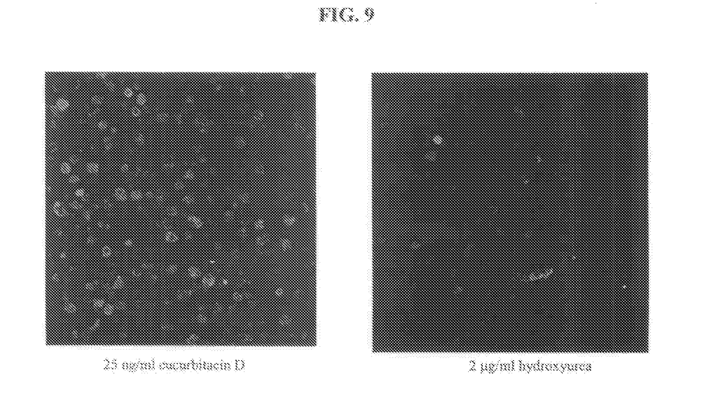

The BM cells were collected from a healthy donor. The mononuclear cells were immediately isolated from the BM cells as method 5.3.1 described. The cells were subsequently cultured as described in example 8. On the other hand, K562 cells treated with cucurbitacin D (12.5 ng/ml) and hydroxyurea (25.0 μg/ml) were cultured for six days. The fetal hemoglobin expression in both studies was analyzed by immunofluorescence confocal microscope as follows. BM mononuclear cells/K562 cells treated with different compounds, at the initial density of 2×10⁴ cells/ml, were cultured in 10 ml cell culture flask for 6 days. An aliquot of 1×10⁶ cells were collected in a 15 ml falcon tube and spun down at 150×g for 5 min. The cell pellet was washed once with 1×PBS, and then the cells were resuspended in 50 μl PBS. An aliquot of 20 μl monoclonal antibody solution was added into the cell suspension. The mixture was incubated for 30 min in dark at 4° C. The cells were washed twice with 1×PBS with 0.1% sodium azide. The cells were resuspended in 0.2 ml 4% formaldehyde in PBS and kept in dark at 4° C. Before confocal microscopic analysis, the cells were mixed thoroughly by vortex. An aliquot of 20 μl cell suspension was transferred on a glass slide, with a cover slip carefully covered on the top. The result indicates cucurbitacin D could induce a higher level expression of fetal hemoglobin than hydroxyurea in BM mononuclear cells (FIG. 8) and in K562 cells (FIG. 9). In FIG. 8, from top row to bottom row, the images are untreated, HU-treated and cucurbitacin D-treated cells, stained by anti-fetal hemoglobin monoclonal antibodies, respectively. The left column is transmission images while the right column is immunofluorescence confocal images.

Manufacturing Pharmaceutical Compositions for Treating Anemia Related Disorders

Once the chemical compound having a desired medical effect is identified in an herb and substantially pure preparations of the compound are obtained either by isolating the compound from natural resources such as plants or by chemical synthesis, various pharmaceutical compositions or formulations can be fabricated from partially purified extract or substantially pure compound using existing processes or future developed processes in the industry. Specific processes of making pharmaceutical formulations and dosage forms (including, but not limited to, tablet, capsule, injection, syrup) from chemical compounds are not part of the invention and people of ordinary skill in the art of the pharmaceutical industry are capable of applying one or more processes established in the industry to the practice of the present invention. Alternatively, people of ordinary skill in the art may modify the existing conventional processes to better suit the compounds of the present invention. The following information is provided for easy reference.

A “pharmaceutically acceptable carrier” is determined in part by the particular composition being administered and in part by the particular method used to administer the composition. A wide variety of conventional carrier may be suitable for pharmaceutical compositions of the present invention and can be selected by people with ordinary skill in the art.

The dose administered to a subject, in the context of the present invention should be sufficient to effect a beneficial therapeutic response in the subject over time. This effective dosage is referred to as “pharmaceutically effective amount.” The effective amount or dosage of an active ingredient can be determined by people skilled in the art.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

### OTHER PUBLICATIONS

Arisawa, M., Pezzuto, J. M., Kinghorn, A. D., Cordell, G. A., Farnsworth, N. R., 1984 Plant anticancer agents. XXX: Cucurbitacins from Ipomopsis aggregata (Polemoniaceae). J Pharm Sci. 73(3): 411-413.
Atweh, G. F., Loukopoulos, D. 2001 Pharmacological induction of fetal hemoglobin in sickle cell disease and beta-thalassemia Semin Hematol. 38: 367-73.
Barbercheck, M. E. and Wang, J., 1996 Effect of cucurbitacin D on in vtro growth of Xenorhabdus and Photorhabdus spp., symbiotic bacteria of entomopathogenic nematodes. J Invertebr Pathol. 68(2): 141-145.
Bean, M. F., Antoun, M., Abramson, D., Chang, C. J., McLaughlin, J. L., Cassady, J. M., 1985 Cucurbitacin B and isocucurbitacin B: cytotoxic components of Helicteres isora. J Nat Prod. 48(3): 500.
Beutler, J. A., McCall, K. L., Herbert, K., Herald, D. L., Pettit, G. R., Johnson, T., Shoemaker, R. H., Boyd, M. R., 2000 Novel cytotoxic ditcpqes from Casearia arborea. J Nat Prod. 63(5): 657-661.
Chen, J. C., Chiu, M. H., Nie, R. L., Cordell, G. A., Qiu, S. X., 2005 Cuctubitacins and cucurbitane glycosides: structures and biological activities. Nat Prod Rep. 22(3): 386-399.
Clericuzio, M., Mella, M., Vita-Finzi, P., Zema, M., Vidari, G., 2004 Cucurbitane triterpenoids from Leucopaxillus gentianeus. J Nat Prod. 67(11): 1823-1828.
Constantoulakis, P., Papayannopoulou, T., Stamatoyannopoulos, G. 1988 α-Amino-N-butyric acid stimulates fetal hemoglobin in the adult. Blood. 72: 1961-7.
DeSimone, J., Heller, P., Hall, L., Zwiers, D., 1982 5-Azacytidine stimulates fetal hemoglobin synthesis in anemic baboons. Proc Natl Acad Sci USA. 79: 4428-31.
Dinan, L., Whiting, P., Girault, J. P., Lafont, R., Dhadialla, T. S., Cress, D. E., Mugat, B., Antoniewski, C., Lepesant, J. A., 1997 Cucurbitacins are insect steroid hormone antagonists acting at the ecdysteroid receptor. Biochem J 327: 643-650.
Dover, G. J., Brusilow, S., Charache, S. 1994 Induction of fetal hemoglobin production in subjects with sickle cell anemia by oral sodium phenylbutyrate. Blood. 84: 339-43.
Duncan, K. L., Duncan, M. D., Alley, M. C., Sausville, E. A., 1996 Cucurbitacin E-induced disruption of the actin and vimentin cytoskeleton in prostate carcinoma cells. Biochem Pharmacol. 52(10): 1553-1560.
Edery, H., Schatzberg-Porath, G., Gitter, S., 1961 Pharmaco-dynamic activity of elatericin (cucurbitacin D). Arch Int Pharmacodyn Ther. 130: 315-335.
Fang, X., Phoebe, C. H., Pezzuto, J. M. Jr., Fong, H. H., Famsworth, N. R., Yellin, B., Hecht, S. M., 1984 Plant anticancer agents, XXXIV. Cucurbitacins from Elaeocarpus dolichostylus. J Nat Prod. 47(6): 988-993.
Fuller, R. W., Cardellina, J. H., Cragg, G. M., Boyd, M. R., 1994 Cucurbitacins: differential cytotoxicity, dereplication and first isolation from Gonystylus keithii. J Nat Prod. 57(10): 1442-1445.
Konoshima, T., Takasaki, M., Kozuka, M., Nagao, T., Okabe, H., Irino, N., Nakasumi, T., Tokuda, H., Nishino, H., 1995 Inhibitory effects of cucurbitane triterpenoids on Epstein-Barr virus activation and two-stage carcinogenesis of skin tumor. II. Biol Pharm Bull. 18(2): 284-287.
Letvin, N. L., Linch, D. C., Beardsley, G. P., McIntyre, K. W., Nathan, D. G. 1984 Augmentation of fetal-hemoglobin production in anemic monkeys by hydroxyurea. N Engl J Med. 310: 869-73.
Liakopoulou, E., Blau, C. A., Li, Q., Josephson, B., Wolf, J. A. 1995 Stimulation of fetal hemoglobin production by short chain fatty acids. Blood. 86: 3227-35.
Musza, L. L., Speight, P., McElhiney, S., Barrow, C. J., Gillum, A. M., Cooper, R., Killar, L. M., 1994 Cucurbitacins, cell adhesion inhibitors from Conobea scoparioides. J Nat Prod. 57(11): 1498-1502.
Papayannopoulou, T., Torrealba de Ron, A., Veith, R., Knitter, G., Stamatoyannopoulos, G. 1984 Arabinosylcytosine induces fetal hemoglobin in baboons by prurbing erytbroid cell differentiation kinetics. Science. 224: 617-9.
Perine, S. P., Miller, B. A., Greene, M. F., Cohen, R. A., Cook N. 1987 Butryic acid analogues augment gamma globin gene expression in neonatal erythroid progenitors. Biochem Biophys Res Commun. 148: 694-700.
Perrine, S. P., Miller, B. A., Faller, D. V., Cohen, R. A., Vichinsky, E. P. 1989 Sodium butyrate enhances fetal globin gene expression in erytliroid progenitors of patients with Hb SS and beta thalassemia. Blood. 74: 454-9.
Rodriguez, N., Vasquez, Y., Hussein, A. A., Coley, P. D., Solis, P. N., Gupta, M. P., 2003 Cytotoxic cucurbitacin constituents from Sloanea zuliaensis. J Nat Prod. 66(11): 1515-1516.
Setzer, W. N., Setzer, M. C., 2003 Plant-derived triterpenoids as potential antineoplastic agents. Mini Rev Med Chem. 3(6): 540-556.
Smit, H. F., van den Berg, A. J., Kroes, B. H., Beukelman, C. J., Quarles van Ufford, H. C., van Dijk, H., Labadie, R. P., 2000 inhibition of T-lymphocyte proliferation by cucmubitacins from Picrorhiza scrophulariaeflora. J Nat Prod. 63(9): 1300-1302.
Stuppner, H. and Wagner, H. 1989 New cucurbitasin glycosides from Picrorhiza kurroa. Planta Med. 55: 559-563.
Torkelson, S., White, B., Faller, D. V., Phipps, K., Pantazis, C. 1996 Erytoid progenitor proliferation is stimulated by phenoxyacetic and phenylalkyl acids. Blood Cells Mol Dis. 22: 150-8.
Veith, R., Papayarnopoulou, T., Kurachi, S., Stamatoyannopoulos, G. 1985 Treatment of baboon with vinblastine: insights into the mechanisms of pharmacologic stimulation of Hb F in the adult. Blood. 66: 456-9.

What is claimed is:

1. A method of treating, preventing, or ameliorating a pathological condition in a mammal, wherein said pathological condition is associated with a pathological deficiency in the oxygen-carrying component of the blood, comprising a step of administering to said mammal a therapeutically effective amount of a cucurbitacin analog.

2. The method of claim 1, wherein said pathological condition is of anemia or hypoxia.

3. The method of claim 1, wherein said pathological deficiency is hemoglobin C disease, hemoglobin S—C disease, sickle cell anemia or a type of thalassemia.

4. The method of claim 1, wherein said cucurbitacin analog is selected from the group consisting of cucurbitacin A, cucurbitacin B, cucurbitacin C, cucurbitacin D, cucurbitacin E, cucurbitacin F, cucurbitacin H, cucurbitacin I, cucurbitacin J, cucurbitacin L, cucurbitacin O, cucurbitacin P, cucurbitacin Q and cucurbitacin S.

5. The method of claim 1, wherein said cucurbitacin analog is a prodrug, bioisostere, N-oxide, deacetylated entity, phanmaceutically acceptable salt or isomer of a compound selected from the group consisting of cucurbitacin A, cucurbitacin B, cucurbitacin C, cucurbitacin D, cucurbitacin E, cucurbitacin F, cucurbitacin H, cucurbitacin I, cucurbitacin J, cucurbitacin L, cucurbitacin O, cucuibitacin P, cucurbitacin Q and cucurbitacin S.

6. The method of claim 4, wherein said cucurbitacin analog is cucurbitacin D.

7. A method of inducing erytrocytic differentiation or hemoglobin expression comprising a step of contacting a red blood cell of a mammalian subject with a cucurbitacin analog.

8. The method of claim 7, wherein said cucurbitacin analog is selected from the group consisting of cucurbitacin A, cucurbitacin B, cucurbitacin C, cucurbitacin D, cucurbitacin E, cucurbitacin F, cucurbitacin H, cucurbitacin I, cucurbitacin J, cucurbitacin L, cucurbitacin O, cucurbitacin P, cucurbitacin Q and cucurbitacin S.

9. The method of claim 7, wherein said cucurbitacin analog is a prodrug, bioisostere, N-oxide, deacetylated entity, pharmaceutically acceptable salt or isomer of a compound selected from the group consisting of cucurbitacin A, cucurbitacin B, cucurbitacin C, cucurbitacin D, cucurbitacin E, cucurbitacin F, cucurbitacin H, cucurbitacin I, cucurbitacin J, cucurbitacin L, cucurbitacin O, cucurbitacin P, cucurbitacin Q and cucurbitacin S.

10. The method of claim 8, wherein said cucurbitacin analog is cucurbitacin D.

11. A method of treating preventing, or ameliorating a pathological condition in a mammal, wherein said pathological condition is associated with a pathological deficiency in the oxygen-carrying component of the blood, comprising a step of administering to said mammal a therapeutically effective amount of an extract from a plant of Trichosanthes.

12. The method of claim 11, wherein said extract is purified to contain substantially a single compound by a purification process which is based on an assay on ingredients' capacity of inducing eryrocytic differentiation or hemoglobin expression.

13. The method of claim 11, wherein said extract is prepared by a process comprising steps of:
(a) extracting said plant of Trichosanthes with a first solvent with a polarity index greater than 2, to afford a liquid extract;
(b) concentrating said liquid extract to form a syrup;
(c) extracting said syrup with a second solvent with a polarity index less than said first solvent to afford a second extract;
(d) concentrating said second extract to form a second syrup; and optionally
(e) drying said second syrup to afford a powder.

14. The method of claim 13, wherein said first solvent is 50-70% ethanol and said second solvent is water.

15. A pharmaceutical composition, comprising a therapeutically effective amount of a cucurbitacin analog and a pharmaceutically acceptable carrier, and being accompanied by a piece of information indicating said pharmaceutical composition is for treating, preventing, or ameliorating a pathological condition in a mammal which is associated with a pathological deficiency in the oxygen-carrying component of the blood.

16. The method of claim 15, wherein said cucurbitacin analog is selected from the group consisting of cucurbitacin A, cucurbitacin B, cucurbitacin C, cucurbitacin D, cucurbitacin E, cucurbitacin F, cucurbitacin H, cucurbitacin I, cucurbitacin J, cucurbitacin L, cucurbitacin O, cucurbitacin P, cucurbitacin Q and cucurbitacin S.

17. The method of claim 15, wherein said cucurbitacin analog is a prodrug, bioisostere, N-oxide, deacetylated entity, pharmaceutically acceptable salt or isomer of a compound selected from the group consisting of cucurbitacin A, cucurbitacin B, cucurbitacin C, cucurbitacin D, cucurbitacin E, cucurbitacin F, cucurbitacin H, cucurbitacin I, cucurbitacin J, cucurbitacin L, cucurbitacin O, cucurbitacin P, cucurbitacin Q and cucurbitacin S.

18. The method of claim 16, wherein said cucurbitacin analog is cucurbitacin D.

19. The method of claim 15, wherein said pathological condition is of anemia or hypoxia.

20. The method of claim 15, wherein said pathological deficiency is hemoglobin C disease, hemoglobin S—C disease, sickle cell anemia or a type of thalassemia.
